(19) United States
(12) Patent Application Publication
(10) Pub. No.: US 2013/0189775 A1
(43) Pub. Date: Jul. 25, 2013

(54) MODULATION OF THE MAMMALIAN RECEPTOR mTOR TO INHIBIT STEM CELL DIFFERENTIATION INTO NEURONS

(71) Applicants: Ye Fang, Painted Post, NY (US); Ronald Allen Faris, Elmira, NY (US); Sadashiva Karnire pai, Painted Post, NY (US); Odessa Natalie Petzold, Elmira, NY (US)

(72) Inventors: Ye Fang, Painted Post, NY (US); Ronald Allen Faris, Elmira, NY (US); Sadashiva Karnire pai, Painted Post, NY (US); Odessa Natalie Petzold, Elmira, NY (US)

(21) Appl. No.: 13/554,597

(22) Filed: Jul. 20, 2012

Related U.S. Application Data
(60) Provisional application No. 61/515,575, filed on Aug. 5, 2011.

(51) Int. Cl. C12N 5/0735; C12N 5/074

(52) U.S. Cl. CPC C12N 5/0606; C12N 5/0607
USPC 435/354; 435/377; 435/366; 435/363; 435/325

(57) ABSTRACT

The present application relates to compositions and methods for inhibiting the differentiation of isolated embryonic stem cells, induced pluripotent stem cells, parthenogentic stem cells, or isolated embryoid bodies into neuronal progenitor cells or neuron cells comprising a mTOR inhibitor and an effective amount of cell culture growth media. The mTOR inhibitor can be Rapamycin.

### CROSS REFERENCE TO RELATED APPLICATIONS

This application claims the benefit of priority under 35 U.S.C. §119 of U.S. Provisional Application Ser. No. 61/515,575 filed on Aug. 5, 2011 the content of which is relied upon and incorporated herein by reference in its entirety.

### BACKGROUND

Neural stem cells exist in the developing brain and in the adult nervous system. These cells can undergo expansion and can differentiate into neurons, astrocytes and oligodendrocytes. Although protocols have been developed for the directed differentiation of stem cells into therapeutically relevant cell types (1), molecular mechanism of differentiation is not very well understood. Further, understanding the molecules involved in the stem cell differentiation into particular type of neurons has both applications for developing assays, substrates for drug discovery, in addition to therapeutic implications.

Various factors and molecules regulate the differentiation of the stem cells into neurons. Extensive studies have shown that epidermal growth factor (EGF), fibroblast growth factor-2 (FGF-2) promote proliferation while retaining the cells in an undifferentiated state (2-5). In addition, insulin and insulin like growth factor 1 (IGF) has also been shown to be essential for neural stem cell growth and proliferation (6-8). Retinoic acid, insulin, IGF, Nogo-66, LPA (lysophosphatidic acid), PTEN (phosphatase and tensin homolog) and Bone morphogenetic protein (BMP)-4 are known to control the differentiation of neural stem cells into neurons or astrocytes (9-16).

The mTOR (mammalian target of rapamycin) plays a central role in controlling protein homeostasis and hence, neuronal functions; indeed mTOR signaling regulates different forms of learning and memory (17). mTOR activity is modified in various pathologic states of the nervous system, including brain tumors, tuberous sclerosis, cortical displasia and neurodegenerative disorders such as Alzheimer's, Parkinson's and Huntington's diseases (18-21). Rapamycin, an FDA approved drug, improves learning and memory and reduces Abeta and Tau pathology (22-23). Recently PTEN/mTOR is shown to be critical for controlling the regenerative capacity of mouse corticospinal neurons (12, 15). Nogo-66, and BMP-4 mediate glial differentiation through STAT-mTOR pathway (13, 14), while insulin or insulin like growth factor (IGF) mediates neural proliferation and differentiation through PI3K-Akt-mTOR activation (3, 9, 11). Conversely LPA is shown to inhibit differentiation of neurons from embryonic stem cells via ROCK-Akt-mTOR pathway (16). For investigators who purposely wish to differentiate stems cells into other cell line lineages other than neurons (e.g., hepatocytes, cardiomyocytes, etc.), it would be advantageous to have compositions and methods available to aide in achieving that purpose.

Various embodiments of the disclosure will be described in detail with reference to drawings, if any. Reference to various embodiments does not limit the scope of the invention, which is limited only by the scope of the attached claims. Additionally, any examples set forth in this specification are not limiting and merely set forth some of the many possible embodiments of the claimed invention.

### SUMMARY

The present application relates to compositions and methods for inhibiting the differentiation of isolated embryonic stem cells, induced pluripotent stem cells, parthenogentic stem cells, or isolated embryoid bodies into neuronal progenitor cells or neuron cells. The composition comprises a mTOR inhibitor and an effective amount of cell culture growth media. Rapamycin or any other inhibitors of mTOR can be used to inhibit neuronal differentiation in culture.

The present application describes and claims a novel method to control and inhibit stem differentiation, and in particular neural stem cell differentiation as neural differentiation is poorly understood and has profound implications in drug discovery and therapeutic implications.

Further provided is a kit for use in preparing a population of isolated stem cells, induced Pluripotent stem cells, parthenogenetic stem cells or isolated embryoid bodies which cannot differentiate into neuronal progenitor cells and/or neurons comprising an effective amount of the composition of a mTOR inhibitor, such as rapamycin.

The present application also describes and claims cell culture devices that are coated with an mTOR inhibitor, such as rapamycin. The mTOR inhibitor is preferably encapsulated inside a control releasing polymeric material. For example, the inhibitor can be incorporated into polymeric microparticles which provide controlled release of the drug(s), wherein the microparticles are used to coat a cell culture device before cell culture. Release of the mTOR inhibitor(s) is controlled by diffusion of the mTOR inhibitor(s) out of the microparticles and/or degradation of the polymeric particles by hydrolysis and/or enzymatic degradation. Suitable polymers include ethylcellulose and other natural or synthetic cellulose derivatives.

### BRIEF DESCRIPTION OF DRAWINGS

FIG. 1 presents representative photographs illustrating the dose dependent inhibition of morphological changes by culture media containing rapamycin; ReN cells were treated with various concentrations of rapamycin alternate days for 6 days in the absence of growth factors, while the control cells did not have any drug; Photomicrographs were taken immediately after treatment.

FIG. 2 presents representative photographs illustrating the dose dependent decrease in β-tubulin III immunostaining in ReN cells treated with culture media containing rapamycin; ReN cells were differentiated by the removal of growth factors and treated with various concentrations of rapamycin except for the control.

FIG. 3 presents representative photographs illustrating that P13-K inhibitors do not inhibit ReN cell morphology changes except wortmannin; ReN cells were differentiated in the absence or presence of various PI3-K inhibitors for 6-7 days. Wortmanninblocked the neural progenitor differentiation, while others did not.

FIG. 4 presents representative photographs illustrating that Akt inhibitors did not inhibit ReN cell differentiation; ReN cells were differentiated in the absence or presence of various Akt inhibitors for 6-7 days. Triciribine killed the cells while other inhibitors like BML-257 and A6730 did not block the neural progenitor differentiation.

FIG. 5 presents representative photographs illustrating that ROCK (Rho kinase) inhibitor (Y27632) did not block neural progenitor cell differentiation. ReN cells were differentiated in the absence or presence of Y27632 (1 μM) for 6-7 days.

FIG. 6 presents graphic representations of a label-free resonant waveguide grating (RWG) biosensor receptor assay profiles illustrating that the neural progenitor cells pretreated with rapamycin blocked the differentiation of cells as evaluated by the RWG biosensor receptor assays; ReN cells differentiated in the absence or presence of rapamycin (5 μM) for 6 days. The label-free biosensor assays for various receptors was done in the presence of various concentrations of a typical agonist using Corning Epic® system. Assay on undifferentiated cells was carried out simultaneously. ReN cells differentiated in the absence of rapamycin showed different profiles compared to the one differentiated in the presence of rapamycin. Undifferentiated receptor profiles were similar to rapamycin treated cells indicating the blockage of differentiation by rapamycin.

### DETAILED DESCRIPTION

### Definitions

The practice of the present invention will employ, unless otherwise indicated, conventional techniques of tissue culture, molecular biology and cell biology, which are within the skill of the art. See, e.g., Sambrook, Fritsch and Maniatis (1989) and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “isolated” is also used herein to refer to cells or tissues that are isolated from other cells or tissues and is meant to encompass both cultured and engineered cells or tissues.

As used herein, “stem cell” defines a cell with the ability to divide for indefinite periods in culture and give rise to specialized cells. At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or 119 (also know as WA01) cell line available from WiCell, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An -induced pluripotent stem cell (iPSC) is an artificially derived stem cell from a non-pluripotent cell, typically an adult somatic cell, produced by inducing expression of one or more stem cell specific genes.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6 (2003).

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, the term “mTOR” refers to a serine/threonine kinase, termed mammalian target of rapamycin, that regulates the expression of proteins involved in cell growth and proliferation via phosphorylation of specific substrates. As such, mTOR plays an integral role in the response to numerous hormones and growth factors. Synonyms for mTOR include FRAP1, FKBP12-rapamycin complex-associated protein, FK506-binding protein 12-rapamycin complex-associated protein 1, rapamycin target protein and RAPT1. Nucleotide and amino acid sequences of mTOR are known in the art (for example, GENBANK™. Accession No. NM.sub.--004958, deposited on Apr. 4, 2002 (SEQ ID NOs: 3 and 4), and GENBANK™ Accession No. BC 117166, deposited on Jun. 26, 2006 (SEQ ID NOs: 5 and 6)).

As used herein, the term “mTOR inhibitor” refers to a molecule that inhibits the activity of mTOR. mTOR inhibitors include, but are not limited to small molecule, antibody, peptide and nucleic acid inhibitors. For example, an mTOR inhibitor can be a molecule that inhibits the kinase activity of mTOR or inhibits binding of mTOR to a substrate. Inhibitors of mTOR also include molecules that down-regulate expression of mTOR, such as an antisense or interference RNA (RNAi). A number of mTOR inhibitors are known in the art and are discussed below. In some embodiments, the mTOR inhibitor is rapamycin or a rapamycin analog such as temsirolimus and everolimus. Other mTOR inhibitors include, but not limited to, KU-0063794, Pp242 and WYE-354.

As used herein, the term “rapamycin” refers to rapamycin and/or structurally modified rapamycin compounds (such structurally modified rapamycin compounds sometimes referred to herein as rapamycin derivatives). The unmodified compound is the macrolide antibiotic that can be produced by Streptomyces hyhoscopius having the structure as disclosed e.g. in J. B. McAlpine et al. J. Antibiotics (1991) 44:688 and S. L. Schrieber et al., J. Am. Chem. Soc., (1991) 113:7433.

As used herein, the term “ReNcell VM” refers to an immortalized human neural progenitor cell line with the ability to readily differentiate into neurons and glial cells. ReNcell VM was derived from the ventral mesencephalon region of a human fetal brain tissue Immortalized by retroviral transduction with the v-myc oncogene, this cell line grows rapidly as a monolayer on laminin with a doubling time of 20-30 hours. Karyotype analyses indicate that the ReNcell VM retains a normal diploid karyotype in culture even after prolonged passage (>45 passages). ReNcell VM was developed by the ReNeuron Group plc, a biotech company that specializes in using human somatic stem cells for therapeutics. In experiments performed by the ReNeuron Group plc, ReNcell VM can be differentiated in vitro to a high level of human dopaminergic neurons. Neurons differentiated from ReNcell VM have furthermore been shown to be electophysiologically active. ReNcell VM may be used for a variety of research applications such as studies of neurotoxicity, neurogenesis, electrophysiology, neurotransmitter and receptor functions.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “composition” is also intended to encompass a combination of active agent and another carrier, (for example, cell culture media). A composition can include such substances as diluent, binder, stabilizer, buffers, salts, lipophilic solvents, preservative, adjuvant or the like. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.). For example, neurons may be identified by expression of neuronal markers B-tubulin III (neuron marker, Millipore, Chemicon), Tuj 1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore, Chemicon); Olig2 (motorneuron marker, Millipore, Chemicon), Olig2 (Millipore, Chemicon), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein); anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter).

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) Brain Res. 1091(1):258-264; Pluchino et al. (2005) Brain Res. Brain Res. Rev. 48(2):211-219; and Goh et al. (2003) Stem Cell Res. 12(6):671-679. Neural stem cells can be identified and isolated by neural stem cell specific markers including, but limited to, CD 133, ICAM-1, MCAM, CXCR4 and Notch 1. Neural stem cells can be isolated from animal or human by neural stem cell specific markers with methods known in the art. See, e.g., Yoshida et al. (2006) Stem Cells 24(12):2714-22. [000X] A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue (see, e.g., Vindigni et al. (2009) Neurol. Res. 2009 Aug. 5. [Epub ahead of print]) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos.: 2009/0263901, 2009/0263360 and 2009/0258421.

A nerve cell that is “terminally differentiated” refers to a nerve cell that does not undergo further differentiation in its native state without treatment or external manipulation. In one embodiment, a terminally differentiated cell is a cell that has lost the ability to further differentiate into a specialized cell type or phenotype

A population of cells intends a collection of more than one cell that is identical (clonal) or non-identical in phenotype and/or genotype.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell, it is preferable to use a positive control (a sample with an aspect that is known to affect differentiation) and a negative control (an agent known to not have an affect or a sample with no agent added).

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

As used herein, a “control release polymeric material” defines a polymeric matrix within which the mTOR inhibitor(s) are encapsulate the mTOR inhibitors, but can be released by diffusion of the inhibitor(s) out of the polymeric matrix and/or degradation of the polymeric matrix by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof. Encapsulation or incorporation of mTOR inhibitors into polymeric carrier materials to produce drug containing microparticles can be achieved through known pharmaceutical formulation techniques.

Cell Culture Generally

Any method of culturing stem cells, pluripotent stem cells and multipotent stem cells known to those of ordinary skill in the art is contemplated for inclusion in the methods of the present invention. standard textbooks and reviews in cell biology, tissue culture, and embryology, including Teratocarcinomas and embryonic stem cells: A practical approach (1987); Guide to Techniques in Mouse Development (1993); Embryonic Stem Cell Differentiation In Vitro (1993); Properties and uses of Embryonic Stem Cells: Prospects for Application to Human Biology and Gene Therapy (1998), all incorporated herein by reference.

Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995).

Growth Media

A variety of media an culture conditions for stem cell culture are known in the art. In certain aspects, cells may be grown with feeder cells such a fibroblasts or in fibroblast conditioned media. However, in some instances it may be preferred that stem cells are grown in the absence of feeder cells. In some aspects, cells may be grown in a defined media such as TeSR (e.g., MTESR™.1 available from BD Biosciences) (Ludwig et al., 2006a, U.S. Application 2006/0084168). Such media may be used for serum free culture of ES cells. In some embodiments, media is supplemented with bovine or human serum to supply the necessary growth factors (Ludwig et al., 2006b).

For example, the culture medium can be DMEM, RPMI 1640, GMEM, or neurobasal medium. The culture medium can contain serum, or can be a serum-free medium. The serum-free medium can be used without the addition of an exogenous growth factor, or can be supplemented with a growth factor such as basic fibroblast growth factor (bFGF), insulin-like growth factor-2 (IGF-2), epidermal growth factor (EGF), fibroblast growth factor 8 (FGF8), Sonic hedgehog (Shh), brain derived neurotrophic factor (BDNF), glial cell line-derived neurotrophic factor (GDNF), or Vitamin C. Similarly, the cells can be grown in suspension on a non-adherent tissue culture surface.

Cell Culture Devices

Examples of devices suitable for cell culture include single and multi-well plates, such as 6, 12, 96, 384, and 1536 well plates, jars, petri dishes, flasks, beakers, plates, roller bottles, slides, such as chambered and multichambered culture slides, tubes, cover slips, bags, membranes, hollow fibers, beads and microcarriers, cups, spinner bottles, perfusion chambers, bioreactors, CellSTACK® (Corning, Incorporated) and fermenters.

Typically, a substrate surface (i.e., layer) will be disposed on a surface of a cell culture device via any known or future developed process. Preferably, the substrate surface provides a uniform layer that does not delaminate during typical cell culture conditions. The substrate surface may be associated with the base material of the device via covalent or non-covalent interactions. Examples of non-covalent interactions that may associate the substrate surface with the base material include chemical adsorption, hydrogen bonding, surface interpenetration, ionic bonding, van der Waals forces, hydrophobic interactions, dipole-dipole interactions, mechanical interlocking, and combinations thereof.

In certain embodiments, mTOR inhibitors are deposited on the substrate surface of a cell culture device. The mTOR inhibitor is diluted in a suitable solvent. Preferably the solvent is compatible with the material forming the cell culture device and substrate surface. It may be desirable to select a solvent that is non-toxic to the cells to be cultured. Alternatively, or in addition, selection of a solvent that can be substantially completely removed or removed to an extent that it is non-toxic. In such circumstances, it may be desirable that the solvent be readily removable without harsh conditions, such as vacuum or extreme heat. Volatile solvents are examples of such readily removable solvents.

Some solvents that may be suitable in various situations for coating articles as described herein include ethanol, isopropanol, acetyl acetate, ethyl acetate, dimethylformamide (DMF), and dimethylsulfoxide (DMSO). In other embodiments, other solvents including acetone, methanol, ethyl acetate, butanone, acetonitrile, 2-propanol and 2-butanol may be applicable.

Inhibition of Neuronal Differentiation

The present application relates to compositions and methods for inhibiting the differentiation of isolated embryonic stem cells, induced pluripotent stem cells, parthenogentic stem cells, or isolated embryoid bodies into neuronal progenitor cells or neuron cells. The compositions comprise a mTOR inhibitor and an effective amount of cell culture growth media. The mTOR inhibitor can be Rapamycin.

More specifically, the inventors have discovered a useful cell culture growth media composition comprising independently the same or different amount of from about 10 nM to about 10 uMM of a mTOR inhibitor.

Still further disclosed and claimed is a composition for inhibiting the differentiation isolated embryonic stem cells, induced pluripotent stem cells, parthenogentic stem cells, or isolated embryoid bodies into neuronal progenitor cells or neuron cells comprising independently the same or different amount of from about 10 nM to about 100 uM of a mTOR inhibitor and an effective amount of cell culture growth media.

The isolated stem cells are of mammalian origin and, in particular, are human cells, simian cells, or murine cells. Isolated embryonic stem cells can be isolated human embryonic stem cells.

Still further, the present application describes and claims an in vitro method for inhibiting the differentiation of one or more of a stem cell selected from the group of isolated embryonic stem cells, induced Pluripotent stem cells, parthenogenetic stem cells or isolated embryoid bodies into a population of neural progenitor cells and/or neurons comprising contacting the cell with about 10 nM to about 100 uM of a mTOR inhibitor. The resulting population is substantially homogenous.

Also, the present application describes and claims a composition for inhibiting the differentiation isolated neuronal stem cells into neuron cells comprising independently the same or different amount of from about 10 nM to about 100 uM of a mTOR inhibitor and an effective amount of cell culture growth media.

Still further, the present application describes and claims a kit for use in preparing a population of isolated stem cells, induced Pluripotent stem cells, parthenogenetic stem cells or isolated embryoid bodies which cannot differentiate into neuronal progenitor cells and/or neurons comprising an effective amount of the composition of claim 1 or 2 and instructions for use of the composition.

Also, the present application describes and claims a cell culture device containing a mTOR inhibitor such as rapamycin. The cell culture device further comprises a surface substrate wherein the mTOR inhibitor is coated onto the surface substrate of the device.

### Description of the Epic System and its Use

Dynamic Mass Redistribution in Cell Signaling and Cell Physiology

Living cells consist of a complex and dynamic network of protein filaments, termed as cytoskeleton which extends throughout the cytoplasm of eukaryotic cells. The cytoskeleton involves in executing diverse cellular activities, for example, by providing tensile strength for maintaining cell shape, by providing the “track” or “docking sites” for signaling and trafficking, and by providing force for cell motion, intracellular transport and cell division. There are three kinds of cytoskeletal filaments: actin filaments, intermediate filaments and microtubules, each executing distinct biological functions. Among these filaments, actin filaments are mostly concentrated just beneath the plasma membrane, as they keep cellular shape, form cytoplasmatic protuberancies, and participate in some cell-to-cell or cell-to-matrix junctions, signal transduction and muscular contraction.

It is known that most of intracellular bio-macromolecules are highly organized by the matrices of filament networks and spatially restricted to appropriate sites in mammalian cells. Furthermore, the localization of cellular proteins is tightly controlled to regulate the specificity and efficiency of protein interactions, to spatially separate protein activation and deactivation mechanisms, and to determine specific cell functions and responses. In response to stimulation, there is often, sometimes significant, relocation of cellular proteins, depending on the nature of signaling pathway and its network interaction, cell status and the cellular context. The relocation of proteins and molecular assemblies is fundamental not only to cell signaling by enabling precise control in its amplitude, duration and kinetics, but also to cell functions such as migration, invasion, growth, cycling, differentiation, survival and death.

Perfect example is illustrated in G protein-coupled receptor (GPCR) signaling GPCRs are a super family of membrane-bound proteins. In unstimulated cells, endogenous GPCRs primarily locate at the cell surface. After being exposed to ligands, cells respond with a series of spatial and temporal events which are tightly and precisely controlled by intracellular signaling and regulatory machineries. These events lead to ordered, directed, directional and dynamic redistribution of cellular contents during the GPCR signaling cycle. Monitoring the dynamic redistribution of cellular contents has provided insights into GPCR signaling and a powerful means for GPCR screens. For example, direct visualization of the relocation of β2-adrenoceptor-GFP conjugates after agonist stimulation initiated interest in this process as a direct screening strategy. One of these trafficking assays is the Transfluor technology from Xsira Pharmaceuticals Inc (formerly Norak Biosciences). This technology employs high resolution fluorescence imaging to monitor the intracellular location of fluorescently labeled arrestins in response to a compound. The re-localization of fluorescently labeled arrestins is viewed as an indication of agonism.

Many of these events occur within the bottom portion of the cells, which can be manifested by optical biosensors, resulting in an optical signal relating to dynamic mass redistribution (DMR). The DMR signals act as novel physiological responses of living cells. Theoretical analysis indicated that the optical signature, as indicated by the shift in incident angle or wavelength, is primarily sensitive to the vertical mass redistribution within the sensing volume (referred to DMR), while the optical output parameters relating to the shape (e.g., peak-width-at-half-maximum, intensity, and area) of the resonant peak are sensitive to the stimulation-induced lateral mass redistribution. Because of the exponential decay of the evanescence wave tail penetrating into the cell layer, a target or complex of certain mass contributes more to the overall response when the target or complex is closer to the sensor surface as compared to when it is further from the sensor surface. Furthermore, the relocation of a target or complex of certain mass towards the sensor surface results in increase in signal, whereas the relocation of a target or complex moving away from the surface leads to decrease in signal.

The DMR signals mediated through a particular target were found to depend on the cell status, such as degree of adhesion, and cell states (such as proliferating and quiescent state). Since the width or position of the resonant peak of a sensor is sensitive to the cell density and viability, the biological status of cells (e.g., cell viability, cell density, and degree of adhesion) that could significantly impact the assay results can be examined, resulting in reduced assay variability.

Three important aspects to qualify the suitability of a given approach for systems biology applications are the ability of multiplexing, of multi-parameter analysis, and of quantitative system-response profiles. Since optical biosensors are label-free and non-invasive, the biosensor-based cell assays are capable of multiplexing. For example, the agonist-induced activation of endogenous bradykinin B₂ receptor, P2Y receptors, as well as protease activated receptors (PARs) in A431 has been found to lead to similar Gq-type optical signatures. Furthermore, since the optical biosensor offers an integrated response, the DMR signaling mediated through a particular target can also be used to profile its downstream signaling target. For example, the EGF-induced DMR in A431 can be used to profile the compounds that target one of its downstream targets: MEK1. These results suggested that the biosensor-based cell assay is multiplexing in nature.

Optical biosensors offer multi-parameters to analyze the ligand-induced DMR responses. These parameters include the shift in angle or wavelength of the reflected light which is sensitive to the vertical mass redistribution, and the parameters defining the shape of the resonant peak which are mostly sensitive to the lateral mass redistribution. Alternatively, since the biosensor is non-invasive, the biosensor-based cell assays can be easily integrated with other technologies, such as mass spectroscopy and fluorescence imaging. These technologies can further confirm the action of compounds or ligands on cells.

The DMR signal mediated through a particular target is an integrated and quantifiable signal that is a sum of contributions from mass redistribution occurring at different distances away from the sensor surface. Because of the complex nature of cell signaling, the activation of distinct cell signaling mediated through different targets might result in similar overall DMR signal. However, because of the participation of unique sets of cellular targets for a specific signaling event, the modulation profiles mediated by a predetermined set of selective modulators might provide a means to classify the specificity of cell signaling being activated by a particular stimulus. Therefore, the DMR response can be treated as a unique and perfect readout for systems biology studies of living cells. These studies showed that the modulations of different targets result in distinct attenuations of the DMR signal induced by EGF. In response to epidermal growth factor (EGF) stimulation, the DMR response of quiescent A431 cells was found to be saturable to the concentration of EGF, and was able to be fully suppressed by a specific and potent EGFR tyrosine kinase inhibitor, AG1478. The effect of various known inhibitors/drugs on the DMR response of quiescent A431 cells linked the cell response to mainly the Ras/mitogen-activated protein (MAP) kinase pathway, which primarily proceeds through MEK.

Cell Assays

Disclosed are the methods and uses of optical free biosensors to perform any cell assay, such as an assay for cell death, an assay for cell proliferation, an assay for receptor activation or inhibition, an assay for cell membrane integrity, an assay for lipid signaling, an assay for cell signaling, an assay for reactive species signaling, an assay for evaluating the redox states of cells, an assay for study cross-communication between distinct targets, an assay for high throughput compound screening using endpoint measurements, and assay nuclear signaling or activity, or an assay for cytoskeleton rearrangement, for example. These assays incorporate the use of one or more biosensor output parameters that can be used to produce a signature for the assay or which can be used to make a determination from the assay as well as one or more steps.

A biosensor can produce an output which can be in different forms, such as pixels, or angular shift, or a wavelength shift, for example. The output can be in the form of a output data which is output information that is collected for a particular set of conditions, and can either be stored or analyzed in realtime. A biosensor when used produces an output of data, which typically can be represented in a graph form, such as response units versus time The response unit can be angular shift (in terms of degree) when an angular interrogation detection system is used, or wavelength shift (in terms of pico-meters when a wavelength interrogation detection system is used), or pixel position shift (in terms of pixel) when an angular interrogation detection system utilizing a CCD camera is used to collect the resonant band images of biosensors. Again, the response unit can be intensity of the incoupled light as a function of incoupling angle or wavelength, when a resonant peak spectrum of a given mode is used. In another embodiment, the response unit can be position in pixel or positional intensity when an angular interrogation detection system utilizing a CCD camera is used to collect the resonant band images of biosensors. A biosensor output parameter or biosensor output data parameter is any characteristic of a biosensor output or output data respectively that can be measured and used to analyze the biosensor output or biosensor output data. In certain embodiments a biosensor output parameter can also be a characteristic that can be identified and used across multiple assays. The signature is any biosensor output parameter or combination of parameters that can be used as a diagnostic for a particular assay. For example, a signature could be the use of peak intensity magnitude after a stimulatory event, or it could be the position of the peak intensity after a stimulatory event, or it could be the position of the half maximal peak intensity. This signature can then be used to, for example, compare the effect of two different compounds on a culture of cells or the effect of a single compound on two different cultures of cells, rather than a comparison of the entire data output. Thus, a signature could occur at a single time point or a single wavelength or single wave angle, or an any combination, depending on what is be assayed.

Biosensor Output Parameters

A number of different biosensor output parameters that can be used. For example, six parameters defining the kinetics of the stimulation-induced directional mass redistribution within the cells can be overall dynamics (i.e., shape), phases of the response (in this specific example, there are three main phases relating to the cell response: Positive-Directional Mass Redistribution (P-DMR), net-zero Directional Mass Redistribution (net-zero DMR) and Negative-Directional Mass Redistribution (N-DMR), kinetics, total duration time of each phase, total amplitudes of both P- and N-DMR phases, and transition time ti from the P- to N-DMR phase. Other biosensor output parameters can be obtained from a resonant peak. For example, peak position, intensity, peak shape and peak width at half maximum (PWHM) can be used. Biosensor output parameters can also be obtained from the resonant band image of a biosensor. The data was obtained using an arrayed angular interrogation system and illustrates 5 five additional features: band shape, position, intensity, distribution and width. All of these parameters can be used independently or together for any given application of any cell assays using biosensors as disclosed herein. The use of the parameters in any subset or combination can produce a signature for a given assay or given variation on a particular assay, such as a signature for a cell receptor assay, and then a specific signature for an EGF receptor based assay.

Parameters Related to the Kinetics of Stimulation-Induced Directional Mass Redistribution

There are a number of biosensor output parameters that are related to the kinetics of the stimulation-induced DMR. These parameters look at rates of change that occurs to biosensor data output as a stimulatory event to the cell occurs. A stimulatory event is any event that may change the state of the cell, such as the addition of a molecule to the culture medium, the removal of a molecule from the culture medium, a change in temperature or a change in pH, or the introduction of radiation to the cell, for example. A stimulatory event can produce a stimulatory effect which is any effect, such as a directional mass redistribution, on a cell that is produced by a stimulatory event. “The stimulatory event could be a compound, a chemical, a biochemical, a biological, a polymer. The biochemical or biological could a peptide, a synthetic peptide or naturally occurring peptide. For example, many different peptides act as signaling molecules, including the proinflammatory peptide bradykinin, the protease enzyme thrombin, and the blood pressure regulating peptide angiotensin. While these three proteins are distinct in their sequence and physiology, and act through different cell surface receptors, they share in a common class of cell surface receptors called G-protein coupled receptors (GPCRs). Other polypeptide ligands of GPCRs include vasopressin, oxytocin, somatostatin, neuropeptide Y, GnRH, leutinizing hormone, follicle stimulating hormone, parathyroid hormone, orexins, urotensin II, endorphins, enkephalins, and many others. GPCRs are a broad and diverse gene family that respond not only to peptide ligands but also small molecule neurotransmitters (acetylcholine, dopamine, serotonin and adrenaline), light, odorants, taste, lipids, nucleotides, and ions. The main signaling mechanism used by GPCRs is to interact with G-protein GTPase proteins coupled to downstream second messenger systems including intracellular calcium release and cAMP production. The intracellular signaling systems used by peptide GPCRs are similar to those used by all GPCRs, and are typically classified according to the G-protein they interact with and the second messenger system that is activated. For Gs-coupled GPCRs, activation of the G-protein Gs by receptor stimulates the downstream activation of adenylate cyclase and the production of cyclic AMP, while Gi-coupled receptors inhibit cAMP production. One of the key results of cAMP production is activation of protein kinase A. Gq-coupled receptors stimulate phospholipase C, releasing IP3 and diacylglycerol. IP3 binds to a receptor in the ER to cause the release of intracellular calcium, and the subsequent activation of protein kinase C, calmodulin-dependent pathways. In addition to these second messenger signaling systems for GPCRs, GPCR pathways exhibit crosstalk with other signaling pathways including tyrosine kinase growth factor receptors and map kinase pathways. Transactivation of either receptor tyrosine kinases like the EGF receptor or focal adhesion complexes can stimulate ras activation through the adaptor proteins Shc, Grb2 and Sos, and downstream Map kinases activating Erk1 and Erk2. Src kinases may also play an essential intermediary role in the activation of ras and map kinase pathways by GPCRs.

It is possible that some stimulatory events can occur but there is no change in the data output. This situation is still a stimulatory event because the conditions of the cell have changed in some way that could have caused a directional mass redistribution or a change in the cell or cell culture.

It is understood that a particular signature can be determined for any assay or any cell condition as disclosed herein. There are numerous “signatures” disclosed herein for many different assays, but for any assay performed herein, the “signature” of that assay can be determined. It is also possible that there can be more than one “signatures” for any given assay and each can be determined as described herein. After collecting the biosensor output data and looking at one or more parameters, or the signature for the given assay can be obtained. It may be necessary to perform multiple experiments to identify the optimal signature and it may be necessary to perform the experiments under different conditions to find the optimal signature, but this can be done. It is understood that any of the method disclosed herein can have the step of “identifying” or “determining” or “providing”, for example, a signature added onto them.

Overall Dynamics

One of the parameters that can be looked at is the overall dynamics of the data output. This overall dynamic parameter observes the complete kinetic picture of the data collection. One aspect of the overall dynamics that can be observed is a change in the shape of the curve produced by the data output over time. Thus the shape of the curve produced by the data output can either be changed or stay steady upon the occurrence of the stimulatory event. The direction of the changes indicates the overall mass distribution; for example, a positive-DMR (P-DMR) phase indicates the increased mass within the evanescent tail of the sensor; a net-zero DMR suggests that there is almost no net-change of mass within the evanescent tail of the sensor, whereas a negative-DMR indicates a net-deceased mass within the evanescent tail of the sensor. The overall dynamics of a stimulation-induced cell response obtained using the optical biosensors can consist of a single phase (either P-DMR or N-DMR or net-zero-DMR), or two phases (e.g., the two phases could be any combinations of these three phases), or three phases, or multiple phases (e.g., more one P-DMR can be occurred during the time course).

Phases of the Response

Another parameter that can observed as a function of time are the phase changes that occur in the data output. A label free biosensor produces a data output that can be graphed which will produce a curve. This curve will have transition points, for example, where the data turns from an increasing state to a decreasing state or vice versa. These changes can be called phase transitions and the time at which they occur and the shape that they take can be used, for example, as a biosensor output parameter. For example, there can be a Positive-Directional Mass Redistribution (P-DMR), a net-zero Directional Mass Redistribution (net-zero DMR) or a Negative-Directional Mass Redistribution (N-DMR). The amplitude of the P-DMR, N-DMR, and the NZ-DMR can be measured as separate biosensor output parameters.

Kinetics

Another biosensor output parameter can be the kinetics of any of the aspects of data output. For example, the rate at the completion of the phase transitions. For example, how fast are the phase transitions completed or how long does it take to complete data output. Another example of the kinetics that can be measured would be the length of time for which an overall phase of the data output takes. Another example is the total duration of time of one or both of the P- and N-DMR phases. Another example is the rate or time in which it takes to acquire the total amplitudes of one or both of the P- and N-DMR phases. Another example can be the transition time τ from the P- to N-DMR phase. The kinetics of both P-DMR and N-DMR events or phases can also be measured. For example, stimulation of human quiescent human epidermoid carcinoma A431 with epidermal growth factor (EGF) results in a dynamic response consisting of at least three phases. The higher the EGF concentration is, the greater are the amplitudes of both the P-DMR and the N-DMR signals, the faster are both the P-DMR and the N-DMR events, and the shorter is the transition time from the P-DMR to the N-DMR event. When the amplitudes of the P-DMR events showed a complicated relationship with the EGF concentrations, the amplitudes of the N-DMR signals were clearly saturable to EGF concentrations, resulting in an EC₅₀ of ˜1.45 nM. The transition time τ in seconds was found to decrease exponentially with the increasing concentration C of EGF. In addition, the decay of the N-DMR signal can be fitted with non-linear regression. The one-phase decay constant κ obtained was also saturable, resulting in a Kd of 5.76 nM.

Parameters Related to the Resonant Peak

Resonant peaks of a given guided mode are a type of data output that occurs by looking at, for example, the intensity of the light vs the angle of coupling of the light into the biosensor or the intensity of the light versus the wavelength of coupled light into the biosensor. The optical waveguide lightmode spectrum is a type of data output that occurs by looking at the intensity of the light vs the angle of coupling of the light into the biosensor in a way that uses a broad range of angles of light to illuminate the biosensor and monitors the intensity of incoupled intensity as a function of the angle. In this spectrum, multiple resonant peaks of multiple guided modes are co-occurred. Since the principal behind the resonant peaks and OWLS spectra is the same, one can use the resonant peak of a given guided mode or OWLS spectra of multiple guided modes interchangeably. In a biosensor, when either a particular wavelength of light occurs or when the light is produced such that it hits the biosensor at a particular angle, the light emitted from the light source becomes coupled into the biosensor and this coupling increases the signal that arises from the biosensor. This change in intensity as a function of coupling light angle or wavelength is called the resonant peak. Distinct given modes of the sensor can give rise to similar resonant peaks with different characteristics. There are a number of different parameters defining the resonant peak or resonant spectrum of a given mode that can be used related to this peak to assess DMR or cellular effects. A subset of these are discussed below.

Peak Position

When the data output is graphed the peak of the resonance peak occurs, for example, at either a particular wavelength of light or at a particular angle of incidence for the light coupling into the biosensor. The angle or wavelength that this occurs at, the position, can change due to the mass redistribution or cellular event(s) in response to a stimulatory event. For example, in the presence of a potential growth factor for a particular receptor, such as the EGF receptor, the position of the resonant peak for the cultured cells can either increase or decrease the angle of coupling or the wavelength of coupling which will result in a change in the central position of the resonant peak. It is understood that the position of the peak intensity can be measured, and is a good point to measure, the position of any point along the resonant peak can also be measured, such as the position at 75% peak intensity or 50% peak intensity or 25% peak intensity, or 66% peak intensity or 45% peak intensity, for example (all levels from 1-100% of peak intensity are considered disclosed). However, when one uses a point other than the peak intensity, there will always be a position before the peak intensity and a position after the peak intensity that will be at, for example, 45% peak intensity. Thus, for any intensity, other than peak intensity, there will always be two positions within the peak where that intensity will occur. The position of these non-peak intensities can be utilized as biosensor output parameters, but one simply needs to know if the position of the intensity is a pre-peak intensity or a post-peak intensity.

Intensity

Just as the position of a particular intensity of a resonant peak can used as a biosensor output parameter, so to the amount of intensity itself can also be a biosensor output parameter. One particularly relevant intensity is the maximum intensity of the resonant peak of a given mode. This magnitude of the maximum intensity, just like the position, can change based on the presence of a stimulatory event that has a particular effect on the cell or cell culture and this change can be measured and used a signature. Just as with the resonant peak position, the resonant peak intensity can also be measured at any intensity or position within the peak. For example, one could use as a biosensor output parameter, an intensity that is 50% of maximum intensity or 30% of maximum intensity or 70% of maximum intensity or any percent between 1% and 100% of maximum intensity. Likewise, as with the position of the intensity, if an intensity other than the maximum intensity will be used, such as 45% maximum intensity, there will always be two positions within the resonant peak that have this intensity. Just as with the intensity position parameter, using a non-maximum intensity can be done, one just must account for whether the intensity is a pre-maximum intensity or a post-maximum intensity.

For example, the presence of both inhibitors and activators results in the decrease in the peak width at half maximum (PWHM) after culture when the original cell confluency is around 50% (at ˜50% confluency, the cells on the sensor surface tend to lead to a maximum PWHM value); however, another biosensor output parameter, such as the total angular shift (i.e., the central position of the resonant peak) can be used to distinguish an inhibitors from an activators from a molecule having no effect at all. The PWHM is length of a line drawn between the points on a peak that are at half of the maximum intensity (height) of the peak. The inhibitors, for example, of cell proliferation, tend to give rise to angular shift smaller than the shift for cells with no treatment at all, whereas the activators tend to give rise to a bigger angular shift, as compared to the sensors having cells without any treatment at all, when the cell densities on all sensors are essentially identical or approximately the same. The potency or ability of the compounds that either inhibit (as inhibitors) or stimulate (as activator) cell proliferation can be determined by their effect on the PWHM value, given that the concentration of all compounds are the same. A predetermined value of the PWHM changes can be used to filter out the inhibitors or activators, in combination with the changes of the central position of the resonant peak. Depending on the interrogation system used to detect the resonant peak of a given mode, the unit or value of the PWHM could be varied. For example, for an angular interrogation system, the unit can be degrees. The change in the PWHM of degrees could be 1 thousandths, 2 thousandths, 3 thousandths, 5 thousandths, 7 thousandths, or 10 thousandths, for example.

Peak Shape

Another biosensor output parameter that can be used is the overall peak shape, or the shape of the peak between or at certain intensities. For example, the shape of the peak at the half maximal peak intensity, or any other intensity (such as 30%, 40%, 70%, or 88%, or any percent between 20 and 100%) can be used as a biosensor output parameter. The shape can be characterized by the area of the peak either below or above a particular intensity. For example, at the half maximal peak intensity there is a point that is pre-peak intensity and a point that is post-peak intensity. A line can be drawn between these two points and the area above this line within the resonant peak or the area below the line within the resonant peak can be determined and become a biosensor output parameter. It is understood that the integrated area of a given peak can also be used to analyze the effect of compounds acting on cells.

Another shape related biosensor output parameter can be the width of the resonant peak for a particular peak intensity. For example, at the width of the resonant peak at the half maximal peak intensity (HMPW) can be determined by measuring the size of the line between the pre-peak intensity point on the resonant peak that is 50% of peak intensity and the point on the line that is post-peak which is at 50% peak intensity. This measurement can then be used as a biosensor output parameter. It is understood that the width of the resonant peak can be determined in this way for any intensity between 20 and 100% of peak intensity

Parameters Related to the Resonant Band Image of a Biosensor

To date, most optical biosensors monitor the binding of target molecules to the probe molecules immobilized on the sensor surface, or cell attachment or cell viability on the sensor surface one at a time. For the binding event or cell attachment or cell viability on multiple biosensors, researchers generally monitor these events in a time-sequential manner Therefore, direct comparison among different sensors can be a challenge. Furthermore, these detection systems whether it is wavelength or angular interrogation utilize a laser light of a small spot (˜100-500 μm in diameter) to illuminate the sensor. The responses or resonant peaks represent an average of the cell responses from the illuminating area. For a 96 well biosensor microplate (e.g., Corning's Epic microplate), each RWG sensor is approximately 3×3 mm² and lies at the bottom of each well, whereas the sensor generally has a dimension of 1×1 mm² for a 384 well microplate format. Therefore, the responses obtained using the current sensor technology only represent a small portion of the sensor surface. Ideally, a detection system should not only allow one to simultaneously monitor the responses of live cells adherent on multiple biosensors, but also allow signal interrogation from relatively large area or multiple areas of each sensor.

Resonant bands through an imaging optical interrogation system (e.g., a CCD camera) are a type of data output that occurs by looking at, for example, the intensity of the reflected (i.e., outcoupled) light at the defined location across a single sensor versus the physical position. Reflected light is directly related to incoupled light. Alternatively, a resonant band can be collected through a scanning interrogation system in a way that uses a small laser spot to illuminate the sensor, and scan across the whole sensor in one-dimension or two-dimension, and collect the resonant peak of a given guided mode. The resonant peaks or the light intensities as a function of position within the sensors can be finally reconsisted to form a resonant band of the sensor. In a biosensor, when either a particular wavelength of light occurs or when the light is produced such that it hits the biosensor at a particular angle, the outcoupled light varies as a function of the refractive index changes at/near the sensor surface and this changes lead to the shift of the characteristics of the resonant band of each sensor collected by the imaging system. Furthermore, the un-even attachment of the cells across the entire sensor after cultured can be directly visualized using the resonant band In an ideal multi-well biosensor microplate, the location of each sensor is relative to normalize to other biosensors; i.e., the sensors are aligned through the center of each well across the row or the column in the microplate. Therefore, the resonant band images obtained can be used as an internal reference regarding to the cell attachment or cellular changes in response to the stimulation. Therefore, such resonant band of each sensor of a given mode provides additional parameters that can be used related to this band to assess DMR or cellular effects. A subset of these are discussed below.

Band Shape

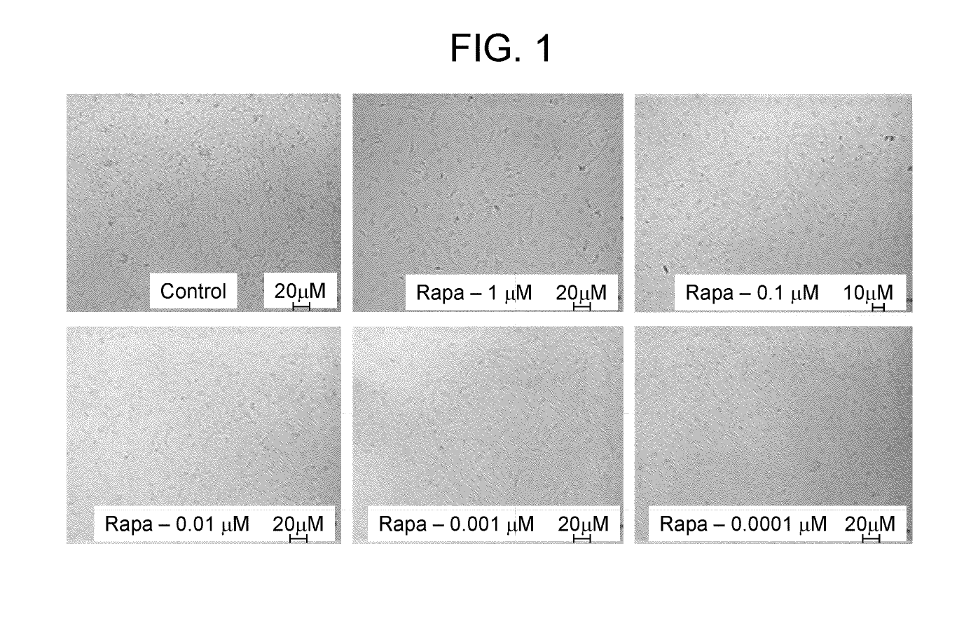

Another biosensor output parameter that can be used is the shape of the resonant band of each biosensor of a given mode. The shape is defined by the intensity distribution across a large area of each sensor. As shown in FIG. 1 and others therein, the shape can be used as an indicator of the homogeneity of cells attached or cell changes in response to stimulation across the large area (for example, as shown in FIG. 1, each resonant band represents responses across the entire sensor with a dimension of ˜200 mm×3000 mm).

Position

Similar to the position of the resonant peak of each sensor of a given mode, the position of each resonant band can be used as a biosensor output parameter. The intensity can be quantified using imaging software to generate the center position with maximum intensity of each band. Such position can be used to examine the cellular changes in response to stimulation or compound treatment.

Intensity

Just as the position of the resonant band, the intensity of the outcoupled light collected using the imaging system can be used as a biosensor output parameter. The average intensity of the entire band or absolute intensity of each pixel in the imaging band can be used to examine the quality of the cell attachment and evaluate the cellular response.

Distribution

The distribution of the outcoupled light with a defined angle or wavelength collected using the imaging system can be used as a biosensor output parameter. This parameter can be used to evaluate the surface properties of the sensor itself when no cells or probe molecules immobilized, and to examine the quality of cell attachment across the illuminated area of the sensor surface. Again, this parameter can also be used for examining the uniformity of compound effect on the cells when the cell density across the entire area is identical; or for examining the effect of the cell density on the compound-induced cellular responses when the cell density is distinct one region from others across the illuminated area.

Width

Just like the PWHM of a resonant peak of a given mode, the width of the resonant band obtained using the imaging system can be used as a biosensor output parameter. This parameter shares almost identical features, thus the useful information content, to those of the PWHM value of a resonant peak, except that one can obtain multiple band widths at multiple regions of the illuminated area of the sensor, instead of only one PWHM that is available for a resonant peak. Similar to other parameters obtained by the resonant band images, the width can be used for the above mentioned applications.

All of these parameters can be used independently or together for any given application of any cell assays using biosensors as disclosed herein. The use of the parameters in any subset or combination can produce a signature for a given assay or given variation on a particular assay, such as a signature for a cell receptor assay, and then a specific signature for an EGF receptor based assay.

Still more aspects of the Epic System and its use are described in U.S. Pat. No. 0,000,000, which is hereby incorporated in its entirety.

### EXAMPLES

The following examples serve to more fully describe the manner of using the above-described disclosure, and to further set forth the best modes contemplated for carrying out various aspects of the disclosure. It is understood that these examples do not limit the scope of this disclosure, but rather are presented for illustrative purposes.

### Example 1

### Cell Culture Experiments

To evaluate the role of kinases in the differentiation of neural stem cells, we added kinase inhibitors from the BIOMOL kinase inhibitor library to the culture during the differentiation process. Kinase inhibitors were replaced on alternate days during media change. Cultures were grown for 6-7 days. We used phase contrast imaging and immunocytochemistry to understand the difference in the morphological differentiation. One of the inhibitors that blocked the differentiation process was Rapamycin, an inhibitor of mammalian target of rapamycin (mTOR).

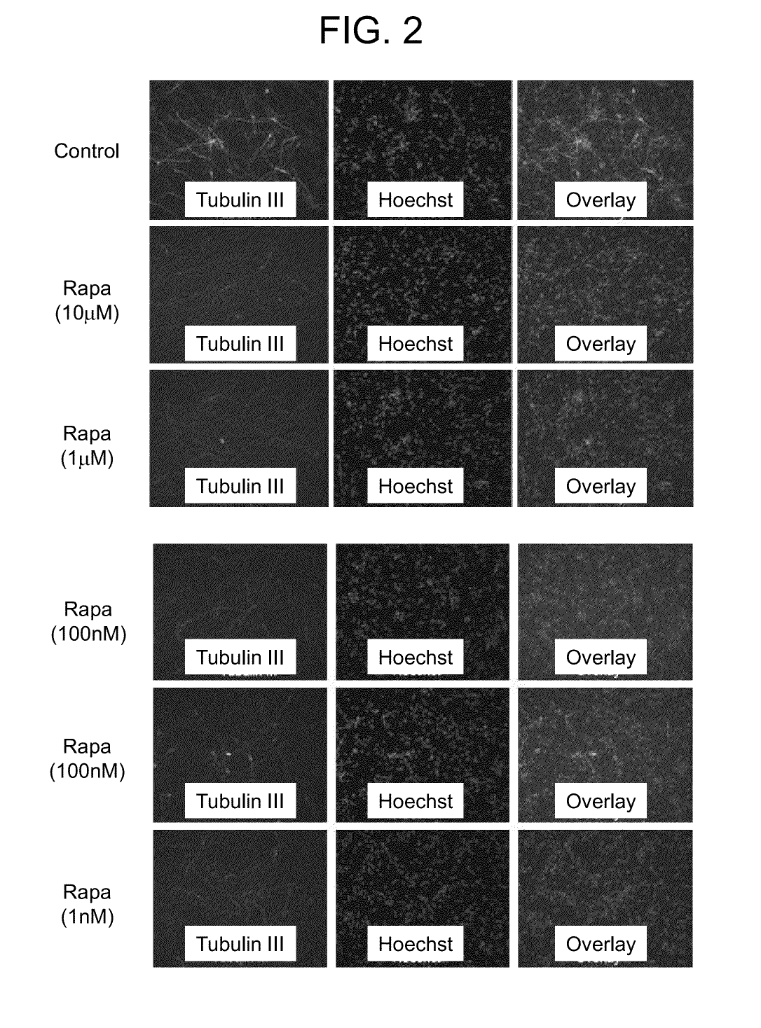

Role of rapamycin was further confirmed by a careful dose response study which clearly demonstrates poor differentiation of neuronal progenitor cells at various concentrations of rapamycin using phase contrast microscopy (FIG. 1) as well as beta-tubulin III staining of neuronal staining (FIG. 2).

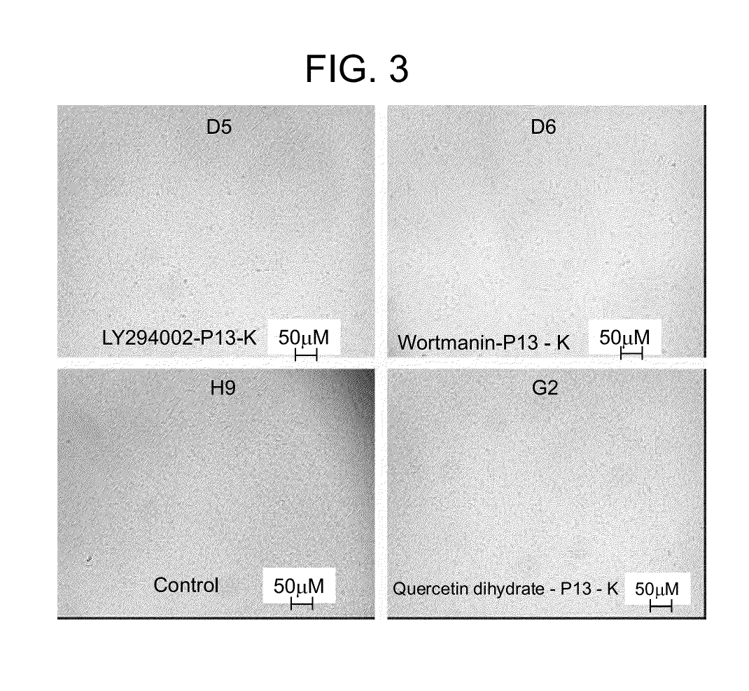

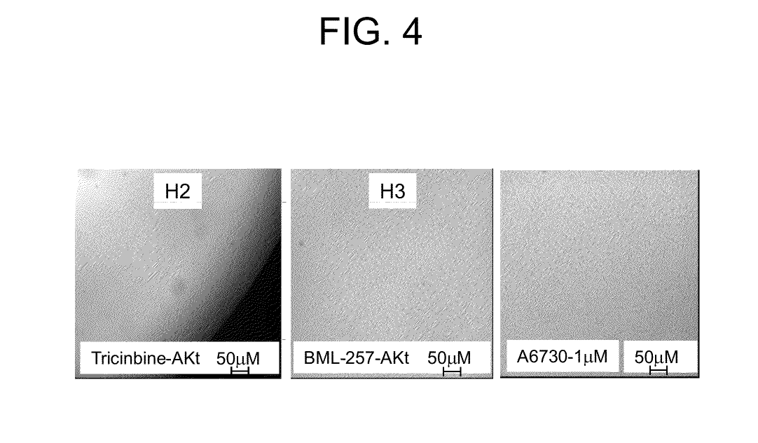

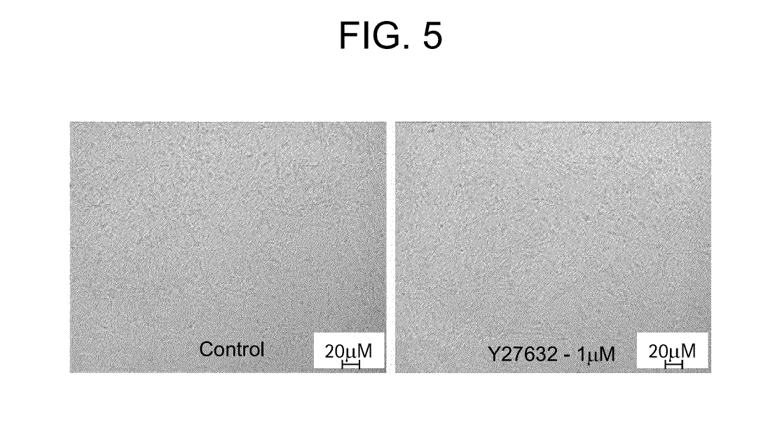

To understand the role of downstream effectors of mTOR, PI3-K, and Akt inhibitors were added culture and the differentiation was monitored. As shown in FIG. 3 various inhibitors of PI3-K other than wortmannin had not effect on the differentiation. Similarly Akt inhibitors tested did not inhibit the differentiation of neuronal progenitor cells (FIG. 4). Rock, inhibitor Y27632 also had no effect on the differentiation process indicating no role for RhoA and ROCK (FIG. 5).

General procedure for neuronal stem cell culture on laminin surface:
96 well CellBind™ treated plates were washed with 70% ethanol and kept ON in the hood
Next day morning, plates were washed twice with 1×PBS
Laminin (50 μl, 20 μg/ml) was added to the wells and incubated 37° C. incubator for 5 hrs.
Wells were washed with 1×PBS after incubation.
Cells were seeded at 20,000 cells/well
For undifferentiated cells, the cultures were grown for 9 days. Media was changed everyday with growth factor supplementation
For differentiated cells, the growth factor deprived media was added after next day after seeding and cells were grown further for 6 days with media changes every other day.
Rapamycin and other inhibitors were added to the cells when the media was changed to growth factor independent media. Media was maintained throughout the study and changed on alternate days.
   ReNcell NSC Maintenance Medium contains DMEM/F12 w/o HEPES, w/L-Glutamine, human serum albumin, human transferrin, putrescine dihydrochloride, human recombinant insulin, L-thyroxine, Tri-iodo-thyronine, progesterone, sodium selenite, heparin, corticosterone. The media does not contain antibiotics.
   The growth factors used were EGF (Epidermal growth factor) and bFGF (Basic fibroblast growth factor).

Screening of BIOMOL Kinase Library

Kinase inhibitors (1 μM) was added to the cells next day after seeding and replenished during media changes on alternate days. Phase contrast microscopy was done after 6 days as described below. The BioMol kinase library consists of 80 kinase inhibitors and was obtained from Enzo Life Sciences. The inhibitors in the library were pre-prepared at 10 mM in 100% DMSO, and used directly after dilution.

Phase Contrast Microscopy

After the experiment, the cells were fixed with 4% paraformaldehyde and washed thrice with 1×PBS. Cells were assessed using a Ziess Axiovert 200M inverted Brightfield/fluorescence microscope.

Immunostaining with β-Tubulin III

After the intended growth period, cells were fixed with 4% paraformaldehyde for 20 min followed by three washes with 1×PBS. Blocking solution containing 1×PBS and 5% donkey serum and Triton X-100 was added and incubated at about 25° C. for 2 hrs. Primary antibodies for 13-tubulin III (Millipore # MAB1637) with appropriate dilutions were added to the cells and incubated for about 16-20 hrs at 4° C. Next, the cells were washed with 1×PBS three times and incubated with Cy3 (Millipore # AP192C) secondary antibody for 1 hr at about 25° C. After washing with 1×PBS twice, the staining of the cells was assessed using a Ziess Axiovert 200M inverted Brightfield/fluorescence microscope using Cy3. Nuclei were stained with Hoechst.

### Example 2

### Epic Assay Confirmation

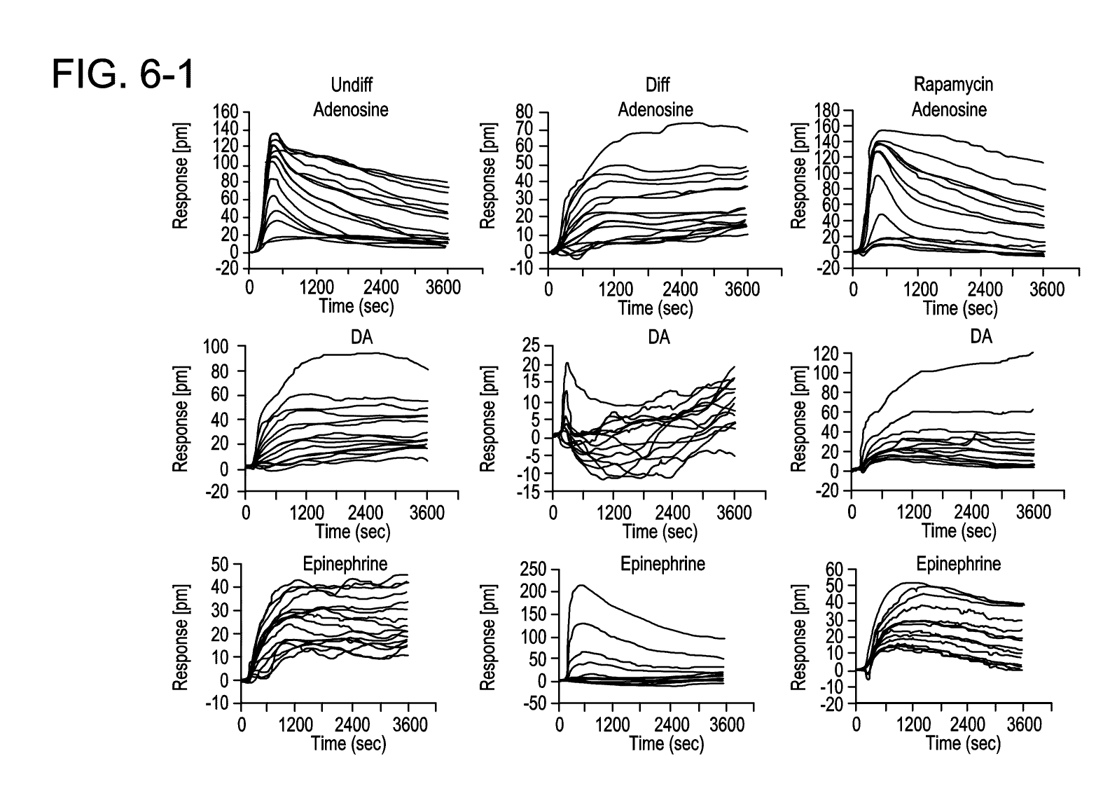

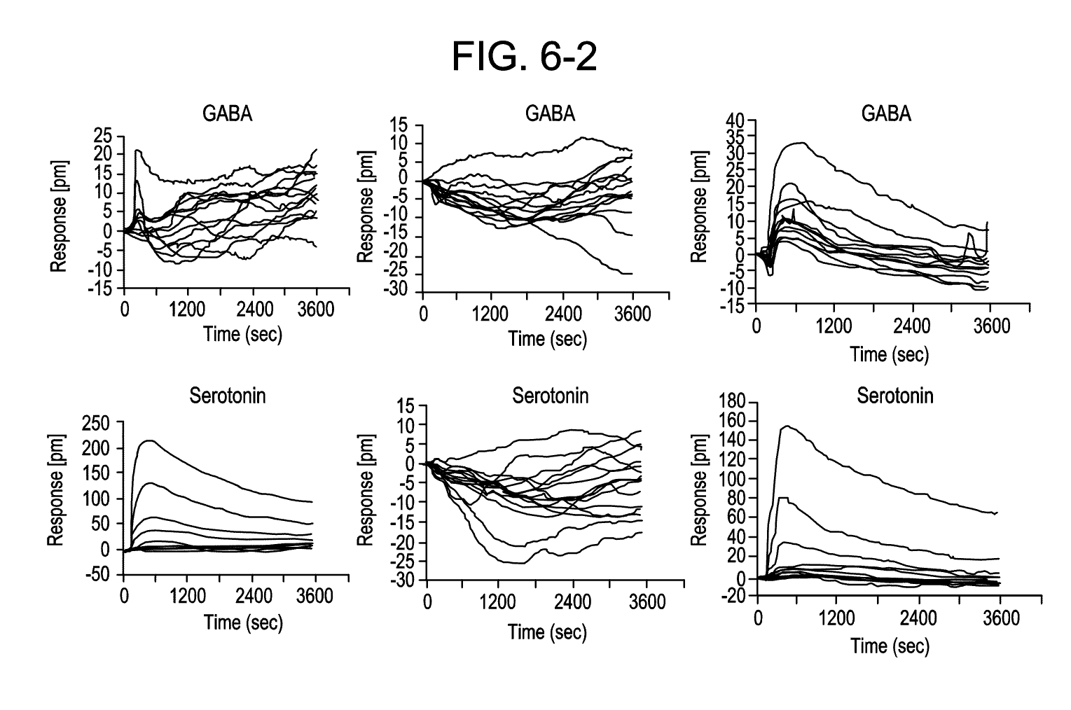

Epic assay was done to further confirm the role of mTOR in the differentiation process of neuronal progenitor cells (FIG. 6). Adenosine, dopamine (DA) and epinephrine showed different profiles on undifferentiated and differentiated cells. Addition of rapamycin maintained the undifferentiated profile indicating cells did not differentiate. Similarly GABA and serotonin showed similar response profile with the undifferentiated and rapamycin treated cells while differentiated cells did not responds to these stimuli.

These results clearly demonstrate the role for mTOR in neuronal progenitor differentiation although the downstream effectors need to be still differentiated and modulating mTOR could control the stem cell differentiation into neurons.

Epic Assay

ReN Cells were treated with or without rapamycin (5 μM) for 6 days. Cells were washed with HBSS and loaded onto Epic instrument for a single step assay with various receptor agonists. Undifferentiated cells grown for 3-4 days (till confluency) and run of Epic instrument as described above.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCES

1 Kim J H, Auerbach J M, Rodríguez-Gómez J A, Velasco I, Gavin D, Lumelsky N, Lee S H, Nguyen J, Sánchez-Pernaute R, Bankiewicz K, McKay R. Dopamine neurons derived from embryonic stem cells function in an animal model of Parkinson's disease. Nature 2002, 418:50-56
2. Ciccolini F, Svendsen C N. Fibroblast growth factor 2 (FGF-2) promotes acquisition of epidermal growth factor (EGF) responsiveness in mouse striatal precursor cells: identification of neural precursors responding to both EGF and FGF-2. J. Neurosci. 1998, 18:7869-7880.
3. Sato A, Sunayama J, Matsuda K, Tachibana K, Sakurada K, Tomiyama A, Kayama T, Kitanaka C. Regulation of neural stem/progenitor cell maintenance by PI3K and mTOR. Neurosci Lett. 2010, 470(2):115-20
4. Vaccarino F M, Ganat Y, Zhang Y, Zheng W. Stem cells in neurodevelopment and plasticity. Neuropsychopharmacology. 2001; 25:805-815.
5. Schwindt T T, Motta F L, Barnabé G F, Massant C G, Guimarães Ade O, Calcagnotto M E, Conceição F S, Pesquero J B, Rehen S, Mello L E. Short-term withdrawal of mitogens prior to plating increases neuronal differentiation of human neural precursor ells. PLoS One. 2009; 4(2):e4642.
6. Arsenijevic Y, Weiss S, Schneider B, Aebischer P. Insulin-like growth factor-I is necessary for neural stem cell proliferation and demonstrates distinct actions of epidermal growth factor and fibroblast growth factor-2. J. Neurosci. 2001, 21(18):7194-2002.
7. Ye P, D'Ercole A J Insulin-like growth factor actions during development of neural stem cells and progenitors in the central nervous system. J Neurosci Res. 2006, 83(1):1-6
8. Erickson R I, Paucar A A, Jackson R L, Visnyei K, Kornblum H. Roles of insulin and transferrin in neural progenitor survival and proliferation. J Neurosci Res. 2008, 86(8):1884-94.
9. Kalluri H S, Vemuganti R, Dempsey R J. Mechanism of insulin-like growth factor I-mediated proliferation of adult neural progenitor cells: role of Akt. Eur J. Neurosci. 2007 February; 25(4):1041-8.
10. Endo M, Antonyak M A, Cerione R A. Cdc42-mTOR signaling pathway controls Hes5 and Pax6 expression in retinoic acid-dependent neural differentiation. J Biol Chem. 2009, 284(8):5107-18.
11. Han J, Wang B, Xiao Z, Gao Y, Zhao Y, Zhang J, Chen B, Wang X, Dai J. Mammalian target of rapamycin (mTOR) is involved in the neuronal differentiation of neuronal progenitor cells induced by insulin. Mol Cell Neurosci. 2008, 39(1):118-24.
12. Liu K, Lu Y, Lee J K, Samara R, Willenberg R, Sears-Kraxberger I, Tedeschi A, Park K K, Jin D, Cai B, Xu B, Connolly L, Steward O, Zheng B, He Z. PTEN deletion enhances the regenerative ability of adult corticospinal neurons. Nat Neurosci. 2010, 13(9):1075-81.
13. Wang B, Xiao Z, Chen B, Han J, Gao Y, Zhang J, Zhao W, Wang X, Dai J. Nogo-66 promotes the differentiation of neural progenitors into astroglial lineage cells through mTOR-STAT3 pathway. PLoS One. 2008, 3(3):e1856
14. Rajan P, Panchision D M, Newell L F, McKay R D. BMPs signal alternately through a SMAD or FRAP-STAT pathway to regulate fate choice in CNS stem cells. J Cell Biol. 2003, 161(5):911-21.
15. Park K K, Liu K, Hu Y, Kanter J L, He Z. PTEN/mTOR and axon regeneration. Neurol. 2010, 223(1):45-50.
16. Dottori A, Leung J, Turnley A, Ebay A. Lysophosphatidic Acid Inhibits Neuronal Differentiation of Neural Stem/Progenitor Cells Derived from Human Embryonic Stem Cells. STEM CELLS 2008; 26:1146-1154
17. Jaworski J, Sheng M. The growing role of mTOR in neuronal development and plasticity. Mol Neurobiol. 2006, 34(3):205-19.
18. Swiech L, Perycz M, Malik A, Jaworski J. Role of mTOR in physiology and pathology of the nervous system. Biochim Biophys Acta. 2008, 1784(1):116-32.
19. Zemke D, Azhar S, Majid A. The mTOR pathway as a potential target for the development of therapies against neurological disease. Drug News Perspect. 2007 October; 20(8):495-9.
20. Ravikumar B, Vacher C, Berger Z, Davies J E, Luo S, Oroz L G, Scaravilli F, Easton D F, Duden R, O'Kane C J, Rubinsztein D C Inhibition of mTOR induces autophagy and reduces toxicity of polyglutamine expansions in fly and mouse models of Huntington disease. Nat Genet. 2004, 36(6):585-95.
21. Pei J J, Hugon J. mTOR-dependent signalling in Alzheimer's disease. J Cell Mol Med. 2008, 12(6B):2525-32.
22. Caccamo A, Majumder S, Richardson A, Strong R, Oddo S. Molecular interplay between mammalian target of rapamycin (mTOR), amyloid-beta, and Tau: effects on cognitive impairments. J Biol Chem. 2010, 285(17):13107-20.
23. Spilman P, Podlutskaya N, Hart M J, Debnath J, Gorostiza O, Bredesen D, Richardson A, Strong R, Galvan V. Inhibition of mTOR by rapamycin abolishes cognitive deficits and reduces amyloid-beta levels in a mouse model of Alzheimer's disease. PLoS One. 2010, 5(4):e9979.

We claim:

1. A composition for inhibiting the differentiation of isolated embryonic stem cells, induced pluripotent stem cells, parthenogentic stem cells, or isolated embryoid bodies into neuronal progenitor cells or neuron cells comprising an amount of an effective cell culture growth media containing a mTOR inhibitor at a concentration of about 10 nM to about 100 μM.

2. The composition of claim 1 containing a mTOR inhibitor at a concentration of about 500 nM to about 5 μM.

3. The composition of claim 1 containing a mTOR inhibitor at a concentration of about 1 μM to about 3 μM.

4. The composition of claim 1 wherein the mTOR inhibitor is selected from the group of rapamycin, KU-0063794, Pp242 and WYE-354, temsirolimus, or everolimus.

5. The composition of claim 1 wherein the stem cells are of mammalian origin.

6. The composition of claim 5 wherein the stem cells are one or more of human cells, simian cells, or murine cells.

7. The composition of claim 1 wherein the isolated embryonic stem cells are isolated human embryonic stem cells.

8. An in vitro method for inhibiting the differentiation of one or more of a stem cell selected from the group of an isolated embryonic stem cell, an induced Pluripotent stem cell, a parthenogenetic stem cell or an isolated embryoid body into a population of neural progenitor cells and/or a neurons comprising contacting an effective amount of a composition of claim 1 with the stem cell, for an effective amount of time, thereby inhibiting the differentiation of the stem cells into a neural progenitor cells or neurons.

9. The method of claim 8, wherein the population is substantially homogeneous.

10. A population of cells produced by the method of claim 8.

11. The method of claim 8, wherein the mTOR inhibitor is selected from the group of rapamycin, KU-0063794, Pp242 and WYE-354, temsirolimus, or everolimus.

12. A cell culture device containing a mTOR inhibitor.

13. The cell culture device of claim 12, wherein the mTOR inhibitor is rapamycin, KU-0063794, Pp242, WYE-354, temsirolimus, or everolimus.

14. The cell culture device of claim 12, wherein the device further comprises a surface substrate.

15. The cell culture device of claim 12, wherein the mTOR inhibitor is encapsulated within a polymeric matrix and can be released over time, wherein the polymeric matrix is coated onto the surface substrate of the device.
